(19) United States
(12) Patent Application Publication
(10) Pub. No.: US 2010/0249702 A1
(43) Pub. Date: Sep. 30, 2010

(54) POROUS CATHETER BALLOON AND METHOD OF MAKING SAME

(75) Inventors: Jesus Magana, Redwood City, CA (US); Randolf von Oepen, Los Altos, CA (US); William E. Webler, Jr., San Jose, CA (US); Allan Bradshaw, Newark, CA (US); Kevin J. Ehrenreich, San Francisco, CA (US)

Correspondence Address: FULWIDER PATTON, LLP (ABBOTT), 6060 CENTER DRIVE, 10TH FLOOR, LOS ANGELES, CA, 90045, US

(73) Assignee: ABBOTT CARDIOVASCULAR SYSTEMS INC., Santa Clara, CA (US)

(21) Appl. No.: 12/409,785

(22) Filed: Mar. 24, 2009

(51) Int. Cl. A61M 25/10; B29C 35/08; B29C 67/20

(52) U.S. Cl. 604/103.01; 264/400; 264/41; 264/49

(57) ABSTRACT

A porous balloon or other catheter structure is formed by creating specific size pores for delivering an agent to a body lumen. The pores can be created by passing matter or energy through the surface of the catheter structure, as by a laser or a projectile. In the case of a laser, the catheter structure can be reversed so that the inner surface becomes the outer surface to convert diverging pores into converging pores. In the case of projectiles, a pore size can be achieved by selecting an appropriate size and shaped projectile to obtain the desired characteristic. Alternatively, a material to make the catheter structure can include impurities that can be removed once the catheter structure is set, leaving pores where the material formed around the impurities.

### BACKGROUND

Treatment of a coronary vessel wall at a treatment site, for regional therapy of vascular disease, includes delivery of a therapeutic agent into the coronary vessel wall. Delivery of therapeutic agents into the coronary vessel wall relies substantially on diffusion of the therapeutic agents through the endothelium into intercellular gaps. Delivery of the therapeutic agents into the coronary vessel wall may be accomplished by, among other things, utilizing drug-effusing balloons at the treatment site. The effused therapeutic agent then migrates into the coronary vessel wall to provide the desired benefit.

The effectiveness of the therapeutic agent into the coronary vessel wall is often limited by the anatomy of the channels within the endothelium, particularly the size of the channels. Endothelial cell gaps and internal elastic lamina gaps are relatively small, and may prevent migration of the therapeutic agents into the vessel wall, since the gaps are smaller than the particles to be introduced. Thus, there is a need for new ways to increase the opportunity for the therapeutic agent to enter the coronary through the endothelial cell gaps, such as by utilizing high pressure to inject the therapeutic agent into the treatment site via an inflatable balloon. The inflated balloon can be delivered to the treatment site where it is inflated to bring the surface of the balloon to bear against the surface of the coronary artery. The therapeutic agent may be allowed to weep through the balloon, or pressure may be employed to impinge the therapeutic agent against the endothelium and thereby force the agents through the cell gaps. The balloon must be porous to effuse the therapeutic agent, and the size of the pores is critical. Moreover, the shape of the pores can play a role in how efficient the delivery of the therapeutic agent is. A pore that is narrower along the interior surface and widens to a larger diameter at the exterior surface will have the effect of decelerating the fluid as it exits the balloon's pores, in contravention of the goal of increasing the fluid's velocity. However, conventional methods of forming pores in a balloon using a laser beam creates the pore described above, i.e., a diverging opening as the fluid passes through the balloon from its interior to the endothelial gaps. The object is to create a converging pore shape, where the fluid would accelerate through the pore due to the narrowing of the pore, creating a jet effect that increases the opportunity for the therapeutic agent to pass through the endothelial cell gaps. Further, the existing laser technologies are capable of forming holes of approximately 10 microns with reasonable manufacturing tolerances and throughput. These balloons are not ideal for the formation of a porous balloon element for the high-speed delivery of therapeutic agents. Hence, a better solution for forming porous balloons that are useful as components of high-speed drug delivery devices are needed.

### SUMMARY OF THE INVENTION

The present invention is a method for forming a porous balloon used in the delivery of therapeutic agents. In a first preferred method of the present invention, a balloon is pierced with a laser in a traditional manner to create a balloon with a plurality of divergent pores across the surface of the balloon. The balloon in then turned inside out by pulling one end of the balloon through an opening until the outer surface becomes the inner surface and the inner surface becomes the outer surface. In this configuration, the divergent pores are converted into convergent pores, which are favored in the delivery of a therapeutic agent because the fluid will accelerate through the pores and impinge the adjacent surface with a higher velocity, increasing the opportunity for penetration of the therapeutic agent into the endothelial cell gaps.

In a second embodiment of the present invention, the size of the pores can be reduced by creating pores in the balloon material by bombarding the balloon surface with projectiles such as spherical particles. This method allows smaller pores to be formed in the balloon than those that are achieved using laser assisted technologies and methods. This method also produces less thermal damage in the balloon material compared with laser methods, preserving the balloon material's inherent strength.

In a third embodiment of the present invention, the pores of the balloon are formed by introducing particles in the balloon material during manufacture that can be removed at a later stage to introduce voids in the material. The particles can be dissolvable, eliminated chemically, or mechanically, to yield a balloon with optimum sized pores that are well controlled and capable of very fine sizes. The small resident pores left behind after the particles are removed provide a passage for the therapeutic agent to be delivered from the balloon's interior to the endothelial cell gaps outside the balloon. Moreover, the size of the pores can be reduced with the present method to coordinate with the therapeutic agent's physical characteristics and the cell gaps' spacing. These and other advantages of the invention will become more apparent from the following detailed description of the invention and the accompanying exemplary drawings.

### BRIEF DESCRIPTION OF THE DRAWINGS

FIG. 1 is an elevated view partially in section of a balloon catheter of the present invention;

FIG. 2 is a transverse cross sectional view of the balloon catheter of FIG. 1 taken along lines 2-2;

FIG. 3 is a transverse cross sectional view of the balloon catheter of FIG. 1 taken along lines 3-3;

FIG. 4 is an enlarged perspective view of the catheter balloon showing the ports;

FIG. 5 is an enlarged perspective view of a laser technique for forming the ports of the balloon;

FIG. 6 is an enlarged sectional view of the port created by the laser technique of FIG. 5;

FIG. 7a is a perspective view of the balloon with ports formed by the technique of FIG. 6;

FIG. 7b is a perspective view partially in shadow of the pulling of a first end of the balloon of FIG. 7a through the internal volume of the balloon;

FIG. 7c is a perspective view of the balloon of FIGS. 7a and 7b as the first end is pulled through and out the second end of the balloon;

FIG. 7d is a perspective view of the balloon of FIG. 7a after the internal and external surfaces have been reversed;

FIG. 8 is an enlarged, sectional view of the ejection port after the reversal of the internal and external surfaces of FIG. 7;

FIG. 9 is an enlarged front view of a projectile just before striking a balloon surface;

FIG. 10 is an enlarged front view of a projectile just after passing through the balloon surface of FIG. 9;

FIG. 11 is an elevated, perspective view of a section of tubing used to form a balloon, where impurities are embedded in the tubing surface;

FIG. 12 is an enlarged, cross sectional view taken along lines 12-12 of FIG. 11;

FIG. 13 is a cross sectional view of a balloon mold and balloon tubing prior to heating and pressurization of the tubing;

FIG. 14 is a cross sectional view of the balloon mold and balloon tubing after heating and pressurization of the tubing to form the balloon, exposing the impurities in the surface of the balloon; and

FIG. 15 is an enlarged, cross sectional view taken along lines 15-15 of FIG. 14 showing the voids left behind after removal of the impurities.

### DETAILED DESCRIPTION OF THE PREFERRED EMBODIMENTS

Regional therapy of vascular disease generally requires the delivery of therapeutic agents into the coronary vessel wall. This can be accomplished in a number of ways. For example, existing technologies such as drug-eluting stents and balloons include the deployment of a medical device coated with a therapeutic agent at the treatment site. The therapeutic agent then migrates into the coronary vessel wall to provide the desired benefit. An obstacle to optimally treating disease with these existing technologies is that the endothelial cell gaps are quite small and often prevent migration of the drug particles, or drugs which are incorporated into a matrix for sustained release, into the vessel wall, since they are smaller relative to the drug particles. Thus, it would be desirable to overcome this issue by injecting the therapeutic agents into, or passing through, the endothelium, thereby creating improved pathways for delivery of the therapeutic agents.

A catheter based system for injecting the therapeutic agents includes an elongate catheter body with a distal and proximal end. A fluid channel spans the length of the catheter body, and is capable of being filled with therapeutic agents for delivery into a vessel wall. The therapeutic agent(s) is delivered rapidly, in a way that creates a jet or blast that can penetrate through the endothelial surface, and into the endothelial cell gaps. This rapid delivery can be driven by a number of methods.

Near the distal end of the catheter body, there is an expandable member that brings the fluid channel proximate to the vessel wall. This expandable member can have a number of forms. In one embodiment, it may be a balloon element, wherein the balloon contains openings throughout the balloon surface, thereby providing injection ports that the therapeutic agent can be delivered through. The opening dimensions are preferably on the order of the endothelial gap size.

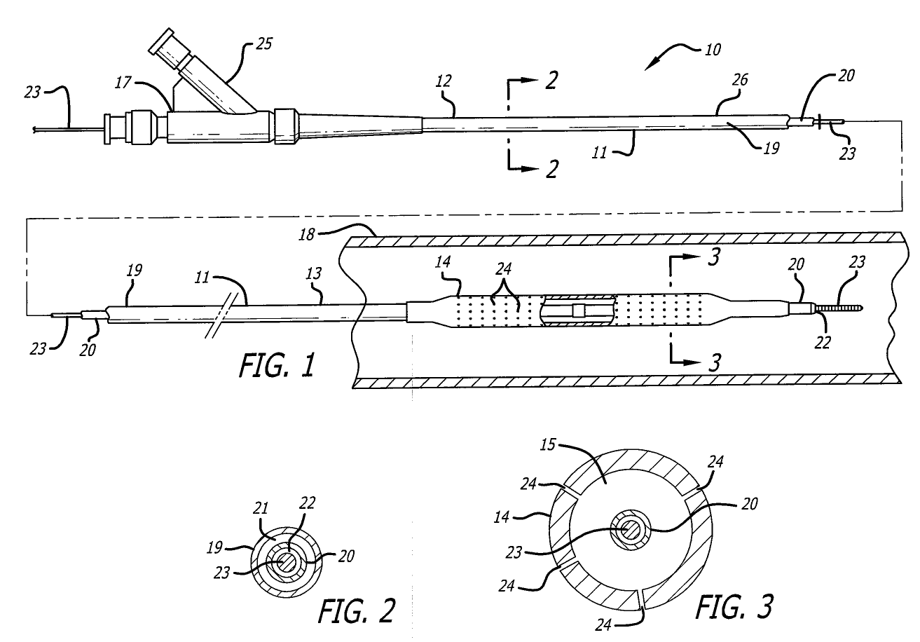

FIG. 1 shows a balloon catheter that can be used to illustrate the features of the invention. The catheter 10 of the invention generally comprises an elongated catheter shaft 11 having a proximal section 12, a distal section 13, an inflatable balloon 14 on the distal section 13 of the catheter shaft 11, and an adapter 17 mounted on the proximal section 12 of shaft 11. In FIG. 1, the catheter 10 is illustrated within a greatly enlarged view of a patient's body lumen 18, prior to expansion of the balloon 14, adjacent the tissue to be injected with therapeutic agents.

In the embodiment illustrated in FIG. 1, the catheter shaft 11 has an outer tubular member 19 and an inner tubular member 20 disposed within the outer tubular member and defining, with the outer tubular member, inflation lumen 21. Inflation lumen 21 is in fluid communication with the interior chamber 15 of the inflatable balloon 14. The inner tubular member 20 has an inner lumen 22 extending therein which is configured to slidably receive a guidewire 23 suitable for advancement through a patient's coronary arteries. The distal extremity of the inflatable balloon 14 is sealingly secured to the distal extremity of the inner tubular member 20 and the proximal extremity of the balloon is sealingly secured to the distal extremity of the outer tubular member 19.

FIGS. 2 and 3 show transverse cross sections of the catheter shaft 11 and balloon 14, respectively, illustrating the guidewire receiving lumen 22 of the guidewire's inner tubular member 20 and inflation lumen 21 leading to the balloon interior 15. The balloon 14 can be inflated by therapeutic agents in a fluid that is introduced at the port in the side arm 25 into inflation lumen 21 contained in the catheter shaft 11, or by other means, such as from a passageway formed between the outside of the catheter shaft and the member forming the balloon, depending on the particular design of the catheter. The details and mechanics of the mode of inflating the balloon vary according to the specific design of the catheter, and are omitted from the present discussion.

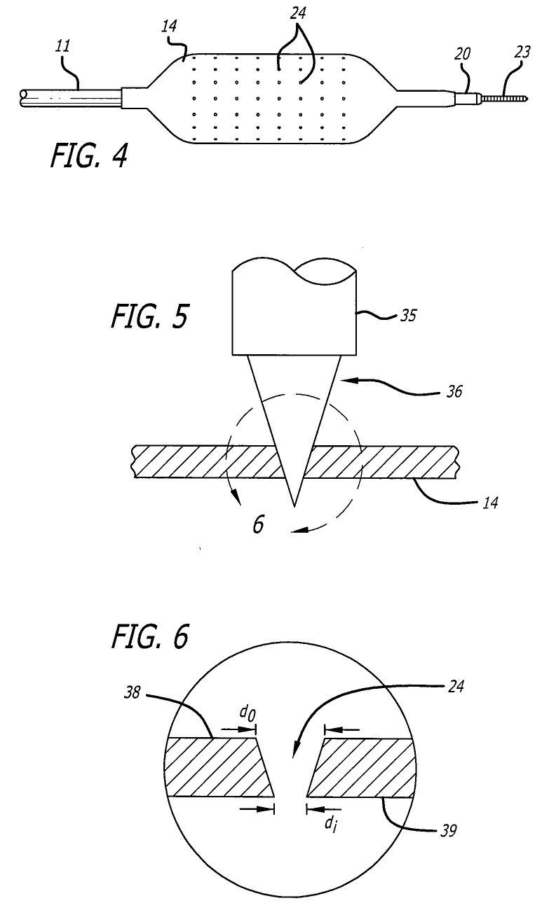

It can be seen that the balloon 14 is porous and includes a plurality of pores 24 throughout the surface of the balloon. FIG. 4 shows an enlarged balloon 14 showing a plurality of pores or “injection ports” 24 through which therapeutic fluids can be dispensed to the coronary vessel 18. The injection ports 24 can have a significant influence on the effectiveness of the therapeutic agents by enhancing the delivery of the agents into the endothelial cell gaps. That is, the jet velocity can be modified by changing the shape of the injection ports 24. Existing technology for creating the injection ports 24 include using a laser source that is directed toward the balloon and focused near the balloon surface. In FIG. 5, a laser source 35 is activated to apply a convergent laser beam 36 into the balloon 14, resulting in a diverging outlet shown in FIG. 6 where the outer diameter do at the outer surface 38 of the balloon is greater than the inner diameter d_{I} at the inner surface 39 of the balloon 14. In this case, the port 24 diverges from the inner volume of the balloon toward the outer wall of the balloon, which acts to slow the fluid velocity in contravention of the goals of effective therapeutic agent insertion into the tissue.

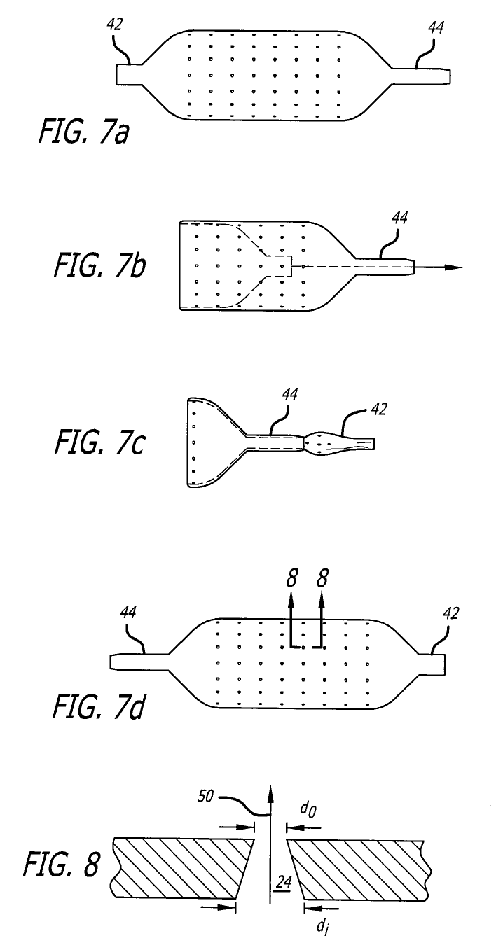

To overcome the shortcomings of the prior balloons, the present invention converts the shortcoming to a benefit as illustrated in FIG. 7 by reversing the inner and outer surfaces to reverse the shape of the injection port. FIG. 7a shows the balloon in the condition of FIG. 4. In FIG. 7b, a proximal end 42 of the balloon 14 is pulled through the balloon's interior, and in FIG. 7c the proximal end 42 is pulled out of the distal end 44 of the balloon. The pulling process is continued until the entire balloon is pulled through the distal end 44, whereupon the balloon will be turned inside out from its original condition as shown in FIG. 7d. It should be noted that the choice of the end for pulling is irrelevant, as the distal end 44 can be pulled through the proximal end 42 to achieve the same result. Also, the selected end can be pushed through the respective opposite end to achieve the same result. The balloon 14 of FIG. 7d has the original inner surface now serving as the outer surface and the original outer surface serving as the inner surface. When the balloon is completely turned inside out as shown in FIG. 7d, the balloon profile will be similar to the original profile prior to reversing the inner and outer surfaces.

An enlarged sectional view of the injection port after reversing the inner and outer surfaces is shown in FIG. 8. It will be appreciated that since the balloon has been reversed, the balloon port profile has also changed. The port now converges rather than diverges from the inner volume toward the outer surface, creating a new profile that will accelerate the fluid exiting the port (along arrow 50) rather than decelerate the fluid. While the outer diameter in FIG. 8 is approximately one half the inner diameter, it is to be understood that any ratio of outer diameter to inner diameter less than one is within the object of the present method. A balloon formed in this manner may be used as a component of a high-speed drug delivery catheter as described above. Further, it may be advantageous to use such a balloon as a component of an infusion balloon or a weeping balloon. The convergence of fluid at relatively low velocities in both of these applications may result in turbulence and flow eddies near the surface of the balloon that improve the activity or delivery of the therapeutic agents.

In addition to the shape of the injection ports, the size of the pores is also a critical factor. Porous balloons used for high-speed delivery of therapeutic agents would benefit from smaller pore diameters. In many applications, the optimum pore size is on the order of 2 to 5 microns because the particle size of the therapeutic agents to be delivered are approximately 1 micron in diameter. As described above, the present method for creating pores in the balloon is through laser cutting or ablation. However, existing laser technologies are only capable of forming hole diameters of approximately ten micron with reasonable manufacturing tolerances and throughput. Therefore, a better method of forming porous balloons is also needed for those applications that would benefit from smaller pore sizes than that which can be obtained using traditional laser technologies.

The present invention contemplates the creation of smaller pores in the balloon using projectiles that are used to bombard the balloon and pierce the balloon to create new pores. This method allows smaller pores to be formed, and can also produce less thermal stress on the balloon material than a laser method. The reduction in thermal stress can preserve the strength of the balloon material as opposed to the laser methodology that can weaken the surrounding material due to thermal stress. As a result, the balloon produced using this methodology is advantageously suited for use as an element of a high-speed drug delivery catheter.

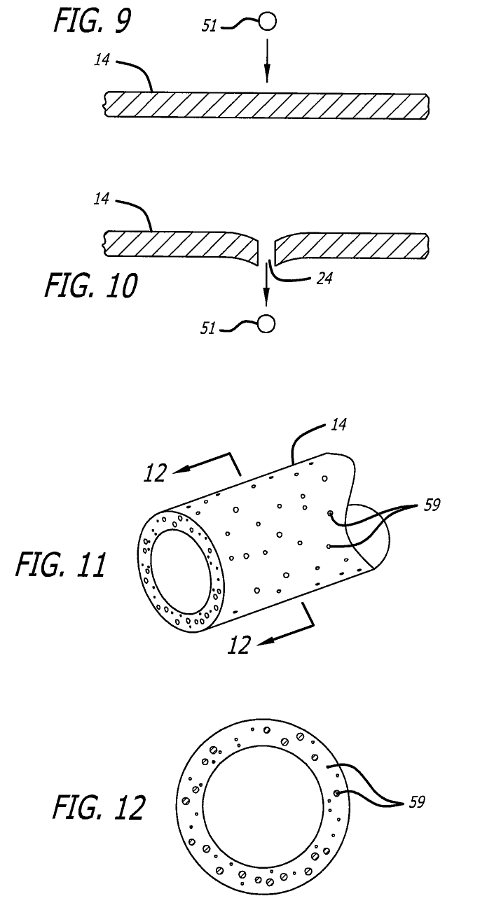

Referring to FIGS. 9 and 10, a method for forming pores in a balloon is disclosed. A section of balloon wall 14 is shown in FIG. 9, and a projectile 51 of an appropriate dimension and material is directed toward the balloon wall at a speed sufficient to pass from one side of the balloon wall to the other side of the balloon wall. As the projectile passes through the wall, it will cause a material elongation and failure, resulting in a hole or pore 24 within the balloon wall 14.

The particles that are delivered toward the balloon wall are contemplated to have the following material and dimensional characteristics. Dimensionally, the particles are to be formed in a relatively spherical configuration. Other shapes are possible, although non-spherical projectiles will lead to inconsistency in the pore dimensions as compared with spherical projectiles. If a greater variation of pore dimension is desired, then non-spherical projectiles or projectiles of varying diameter would be beneficial. The projectiles 51 preferably have a diameter of approximately 80%-120% of the diameter of the intended pore size. For example, for a desired pore size of 2 microns, the projectile will have a diameter of approximately 1.6 to 2.4 micron. The projectiles can be formed from a material with a viscoelastic time coefficient that is greater than that of the balloon material. This will result in a propensity for the particles to pass through the balloon as they impact the balloon wall, rather than being compressed and deflected or embedded within the balloon surface. As an example, the projectiles may be formed from a metal such as gold or silver, which are relatively stiff compared with typical balloon materials such as polyvinyl chloride, polyethylene terephthalate, nylon, and Pebax.

The projectile may also have a core of a material with a higher viscoelastic time coefficient material than the balloon material, but at least one layer around the core of the projectile is formed from a material that has a lower viscoelastic time coefficient. For example, a gold core projectile may be coated with a lubricious gel or fluid. The gel coating will slough off as the projectile penetrates through the material and thereby lubricate the particle path. This lubrication reduces the friction between the projectile and the balloon, making it easier for the projectile to completely pass through the balloon wall with minimal distortion.

Various modes can be employed for emitting the projectiles toward the balloon surface. In one embodiment, the projectiles may be accelerated along a tube either directly or indirectly (via an intermediate membrane) by a pneumatic flow. The projectiles will eject from the tube near the balloon surface and thereby impinge and penetrate the balloon material. In another embodiment, the projectiles may originally be associated with a surrounding sheath formed from a material that is capable of being ablated by a laser. Ablation of the sheath from an opposite surface will create a thermal and/or pressure shock wave that propagates toward the projectile laden surface and ejects the projectiles from the surface toward the balloon material. Other means are also available for accelerating the projectiles toward the balloon material to form the pores 24 in accordance with the invention.

The resulting balloon is suitable for use in many medical device applications that require a porous balloon. For example, the balloon could be used to weep therapeutic agents into a patient's vasculature. Also, such a balloon may be used as an element of a high-speed drug delivery device for injecting therapeutic agents into the endothelial cell gaps as discussed above, as the small pore size can be utilized to increase the velocity of the jets emitting from the balloon. The method of the invention is not limited to balloons, as other parts of the catheter can be impregnated with pores using the above described method to produce a weeping-type catheter body or suction ports within a catheter body. Alternatively, it can be used to create small orifices in a catheter such as a guidewire port or other port of a size below that which is available using other catheter forming techniques.

An alternate method of forming a balloon with pore sizes smaller than that available with traditional laser techniques is to impregnate the balloon material with particles or impurities during the formation of the balloon. The impurities are intentionally included in the material so that they can be later removed to create voids in the balloon. Once the balloon is formed with the impurities in the balloon wall and the material has set, the balloon is expanded and the impurities are removed from the balloon material either through physical migration, mechanical means, thermal means, chemical means, or other mean to create voids in the balloon material that serve as ports through which fluid can pass.

Referring to FIG. 11, the tubing that the balloon 14 is formed of is shown as including solid particles 59 embedded in the surface. These deposits can be formed by mixing in particles during the extrusion process or prior to the formation of the tubing. It will be appreciated that many different types of particles 59 can be used for the present method. In a first preferred method, the particles are soluble such as water soluble materials like salt or sugar. In the case of soluble particles, the tubing can be exposed to water or other solvents to dissolve the particles and thereby leave a void in the material. Alternatively, silicate particles can be embedded into the surface of the tubing to form the material impurities.

In addition to solid impurities, other impurities can be used with the present invention. For example, localized bubbles can be formed by injecting a gas into the material just prior to or during the extrusion process. The bubbles would result in localized material displacement during expansion of the balloon, creating the pores needed to carry out the invention. FIG. 12 shows a cross-sectional view of the extruded tubing taken about line 12-12 of FIG. 11. The randomly dispersed impurities create localized changes in the material strength and, in a preferred embodiment, bond poorly with the surrounding tubing material. This latter characteristic ensures that the impurities will easily be dislodged when the balloon is expanded and migrate out of the material. As shown, the impurities 59 may have varied size, shape, and characteristics as dictated by the particular application.

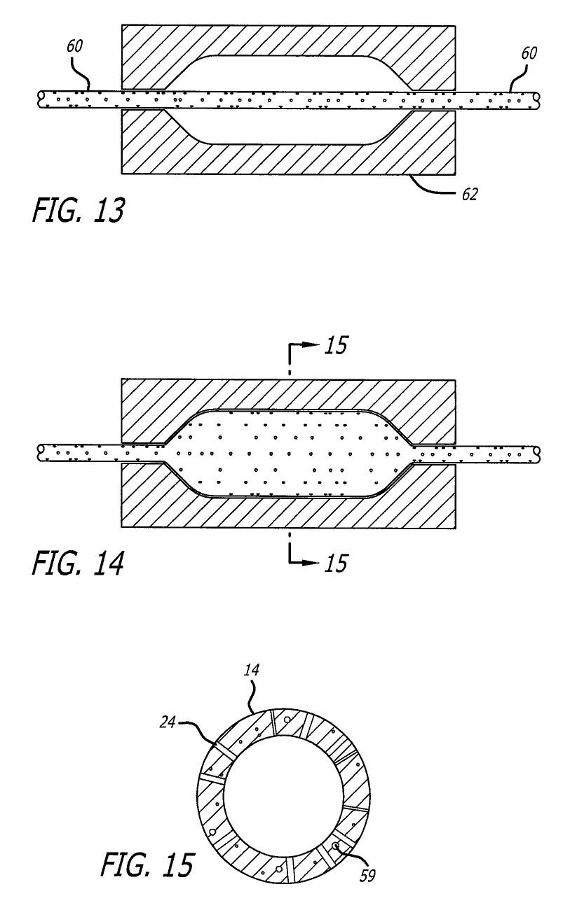

The tubing is formed into a balloon using conventional balloon technologies, such as that illustrated in FIGS. 13 and 14. The tubing 60 is inserted into a mold 62 having the desired balloon shape. The balloon material is then heated and pressurized to cause the tubing 60 to expand to the final shape within the mold 62. As the tubing 60 is expanded toward the balloon shape, the balloon wall is required to stretch and expand, as well as to become more thin. As this material deformation occurs, the balloon material surrounding the impurities will deflect away from the impurities, leaving the impurities free to be expelled from the material. When this occurs, the impurities will be removed passively or actively out of the balloon material such as by solvents, air stream, ultrasonic cleaning, vacuum, etc. Voids left behind by the removed impurities create the pores of the desired balloon. FIG. 15 is a cross sectional view of the balloon of FIG. 14 taken along line 15-15. The balloon layer contains pores remaining where the impurities have dislocated, and the pores can be loaded with therapeutic agents and ejected at a high speed into the vascular wall as part of a high-speed drug delivery device. Alternatively, balloons formed in this manner may also be useful for drug infusion or weeping therapeutic agents into a patient's vascular.

While particular forms of the invention have been illustrated and described, it will be apparent to those skilled in the art that various modifications can be made without departing from the spirit and scope of the invention. Accordingly, it is not intended that the invention be limited except by the appended claims

1. A method for forming pores in a tubular structure of a catheter comprising:
directing a convergent laser beam through a surface of the tubular structure to create a pore diverging from an inner surface to an outer surface of the tubular structure; and
passing a first end of the tubular structure through the tubular structure to reverse the inner surface into the outer surface;
whereby the reversing of the inner surface into the outer surface converts the diverging pore into a converging pore.

2. The method of claim 1 wherein the catheter tubular structure is an inflatable balloon.

3. The method of claim 1 wherein a size of the pore is selected to emit therapeutic agents as the catheter is placed in a body lumen.

4. The method of claim 1 wherein an outer diameter of the converging pore is approximately one half the inner diameter of the converging pore.

5. The method of claim 1 wherein a size of the pore is selected to ballistically deliver a drug to a body lumen.

6. The method of claim 1 wherein a size of the pore is selected to weep a drug to a body lumen.

7. A catheter balloon having a plurality of pores disposed across an outer surface, the pores having an inner diameter at an inner surface of the balloon and an outer diameter at an outer surface of the balloon, where the outer diameter is approximately one half of the inner diameter.

8. A method for forming pores in a tubular structure of a catheter comprising:
providing a plurality of projectiles having a diameter of approximately 80% to 120% of a desired pore size for the tubular structure; and
shooting the projectiles through a surface of the tubular structure at a speed to pass the projectiles from one side of the balloon surface to an opposite surface to form holes in the tubular structure.

9. The method of claim 8 wherein the projectiles are spherical.

10. The method of claim 8 wherein the projectiles are not spherical.

11. The method of claim 8 wherein the projectiles are of varying diameters.

12. The method of claim 8 wherein the holes formed in the tubular structure have a diameter of approximately 2 to 5 microns.

13. The method of claim 8 wherein the projectiles are coated with a viscoelastic material.

14. The method of claim 8 wherein the diameter of the projectiles is approximately between 1.6 and 2.4 microns.

15. The method of claim 8 wherein the projectiles are formed of a metal selected from gold and silver.

16. The method of claim 8 wherein the tubular structure is selected from a group comprising polyvinyl chloride, polyethylene terephthalate, nylon, and Pebax.

17. The method of claim 8 wherein the projectile has a core of a material with a higher viscoelastic time coefficient material than a material forming the tubular structure, but at least one layer around the core of the projectile is formed from a material having a lower viscoelastic time coefficient than the material forming the tubular structure.

18. The method of claim 8 wherein the tubular structure is a catheter balloon.

19. The method of claim 8 wherein the projectiles are accelerated toward the surface of the tubular structure using a pneumatic flow.

20. The method of claim 8 wherein the projectiles are accelerated toward the surface of the tubular structure via a laser ablatable material.

21. A method for forming pores in a tubular structure of a catheter comprising:
introducing into a material used to form the tubular structure impurities that can be removed from the tubular structure after the tubular structure has been formed;
forming the tubular structure using the material with the impurities, and allowing the material to set;
removing the impurities after the material has set to leave pores in the material of a size corresponding to the impurities removed from the material.

22. The method of claim 21 wherein the tubular structure is a catheter balloon.

23. The method of claim 22 wherein the impurities are removed from the balloon by inflating the balloon after the material with the impurities used to form the balloon has set.

24. The method of claim 21 wherein the impurities are removed mechanically.

25. The method of claim 21 wherein the impurities are removed thermally.

26. The method of claim 21 wherein the impurities are removed chemically.

27. The method of claim 21 wherein the impurities are soluble.

28. The method of claim 27 wherein the impurities is selected from salt and sugar.

29. The method of claim 21 wherein the impurities are removed by exposure to water.

30. The method of claim 21 wherein the impurities are removed by exposure to a solvent.

31. The method of claim 21 wherein the impurities are gaseous and form bubbles in the material.

32. The method of claim 21 wherein the impurities are selected to bond poorly with the material forming the tubular structure.

33. The method of claim 21 wherein the impurities have different shapes.

34. The method of claim 21 wherein the impurities have different sizes.

35. The method of claim 21 wherein the tubular structure is heated and expanded in a mold to form a balloon shape.

36. The method of claim 21 wherein the impurities are removed by applying an air stream to the tubular structure.
